(19) United States
(12) Patent Application Publication
(10) Pub. No.: US 2009/0004240 A1
(43) Pub. Date: Jan. 1, 2009

(54) IMPLANTS WITH A PHOSPHAZENE-CONTAINING COATING

(75) Inventors: Olaf Fritz, Hirschhorn (DE); Ulf Fritz, Hirschhorn (DE); Stefan Nagel, Hennigsdorf (DE); Michael Boxberger, Berlin (DE)

Correspondence Address: SUTHERLAND ASBILL & BRENNAN LLP, 999 PEACHTREE STREET, N.E., ATLANTA, GA, 30309, US

(73) Assignee: CeloNova BioSciences, Inc., Newnan, GA (US)

(21) Appl. No.: 12/111,370

(22) Filed: Apr. 29, 2008

Related U.S. Application Data
(63) Continuation-in-part of application No. 10/344,216, filed on Apr. 11, 2003.

(30) Foreign Application Priority Data
Aug. 11, 2000 (EP) 00117191.7

(51) Int. Cl. A61F 2/00; A61K 39/395; A61K 38/18; A61K 48/00; A61K 31/095; A61K 31/4418; A61P 7/02; A61K 31/74

(52) U.S. Cl. 424/423; 424/141.1; 514/12; 514/44; 514/712; 514/277; 424/78.08

(57) ABSTRACT

The present disclosure relates to implants with a biocompatible coating having antithrombogenic properties and which also contains a pharmacologically active agent, as well as a process for their production.

### CROSS-REFERENCE TO ELATED APPLICATIONS

This application is a continuation in part of U.S. patent application Ser. No. 10/344,216, which is a National Stage Entry of PCT/EP2001/08913, filed Aug. 1, 2001, which claims priority to European Patent Application No. EP 00117191.7, filed Aug. 11, 2000, all entitled “Implants with a Phosphazene-Containing Coating”, and the entire disclosures of which are incorporated herein by reference.

### BACKGROUND OF THE INVENTION

The present disclosure relates to artificial implants with a biocompatible coating having antithrombogenic properties and which also contains a pharmacologically active agent, as well as a process for their production.

The most serious complications caused by artificial implants are considered to be the increased deposition of thrombocytes on the exogenous surface. Such thrombi formation on contact of human blood with the exogenous surface, such as artificial heart valves, is described at the state of the art (cf information material from the company Metronic Hall, Bad Homburg, Carmeda BioActive Oberflche [Carmeda BioActive Surface] (CBSA), pages 1-21; B. D. Ratner, “The Blood Compatibility Catastrophe”, J. of Biomed. Mat. Res., Vol. 27, 283-287; and C. W. Akins, “Mechanical Cardiac Valvular Prostheses”, The Society of Thoracic Surgeons, 161-171 (1991)). For example, artificial heart valves found on the world market are made of pyrolyzed carbon and exhibit an increased tendency for development of thrombi (cf. C. W. Akins, above).

The polymeric compound poly[bis(trifluoroethoxy)phosphazene] was used to coat artificial implants in DE-C-19613048. Its effective antithrombogenic action was known from Holleman Wiberg, “Stickstoffverbindungen des Phosphors” [Nitrogen Compounds of Phosphorus], Lehrbuch der anorganischen Chemie [Textbook of Inorganic Chemistry], 666-669, 91^{st}-100^{th} Edition, Walter de Gruyter Verlag (1985), and from Tur, Vinogradova, et al., “Entwicklungstendenzen bei Polymeranalogen Umsetzungen von Polyphosphazen” [Tendencies in development of polymer-like reactions of polyphosphazenes], Acta Polymerica 39, 424-429, No. 8, (1988). Specifically, DE-C-19613048 describes an artificial implant comprising an implant material as the substrate and a biocompatible coating applied at least partly to the surface of the substrate, which coating contains an antithrombogenic polymer having the following general formula (I):

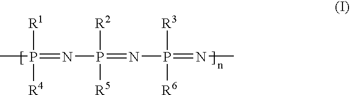

wherein R¹ to R⁶ are the same or different and represent an alkoxy, alkylsulfonyl, dialkylamino or aryloxy group, or a heterocycloalkyl or heteroaryl group having nitrogen as the heteroatom; it also describes methods of producing such artificial implants.

A problem with implants such as heart valves and stents (see DE-A-197 53 123), independently of whether the implant is coated with the present antithrombogenic material, is their tendency to restenosis, i.e., narrowing due to proliferation of smooth muscle cells in the vessel wall as a biological response to the implant. A survey article by Swanson and Gershlick (Stent, Vol. 2, 66-73 (1999)) mentions numerous approaches to the application of suitable active agents to the implants. These include the use of polymer-coated stents, suggested on page 68, wherein the polymer can act as a reservoir for active agents. However, it is immediately advised that this approach not be pursued, because an elevated tendency to inflammation was found in vivo in a test study in which stents were coated with various biodegradable polymers, all of them otherwise known to be biocompatible in vitro. Furthermore, U.S. Pat. Nos. 5,788,979 and 5,980,972 describe coating of materials with biodegradable polymers, in which the coating can also contain pharmacologically active agents.

An alternative approach to preventing excessive cell proliferation and the formation of scars is described in WO 99/16477. In this case, a radioactively labeled polymer of formula (I), above, preferably a polymer containing a radioactive isotope of phosphorus, is applied to the implant. The radioactive radiation emitted (β-radiation with ³²P) is said to prevent uncontrolled cell growth, which results in restenosis on stent implantation, for instance. Of course, when radioactive materials are used, safety requirements and side effects must be considered that stand in the way of the straightforward use of such implants.

### BRIEF SUMMARY OF THE INVENTION

One aspect of the present disclosure provides for artificial implants having not only outstanding mechanical properties but also antithrombogenic and anti-restenosis properties so as to improve the biocompatibility and tolerability of such implants.

In a further aspect, the present disclosure provides processes for the production of such implants.

### DETAILED DESCRIPTION OF THE INVENTION

Embodiments of devices and methods disclosed herein comprise certain polyphosphazene polymers having the following general formula (I),

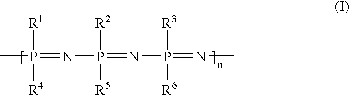

wherein R¹ to R⁶ are the same or different and are selected independently from an alkoxy, an alkylsulfonyl, a dialkylamino, an aryloxy, a heterocycloalkyl having nitrogen as the heteroatom, a heteroaryl group having nitrogen as the heteroatom, or a halogenated derivative thereof, any of which independently having up to 20 carbon atoms, and wherein n may vary from at least about 1 to ∞, as defined herein. Typically, n may vary from about 40 to about 100,000. In one aspect, for example, R¹ to R⁶ are all trifluoroethoxy (OCH₂CF₃) groups, and n may vary from about 40 to about 100,000. Alternatively, one may use derivatives of this polymer in the present invention. The term “derivative” or “derivatives” is meant to refer to polymers made up of monomers having the structure of formula I but where one or more of the R¹ to R⁶ functional group(s) is replaced by a different functional group(s), such as an unsubstituted alkoxide, a halogenated alkoxide, a fluorinated alkoxide, or any combination thereof, or where one or more of the R¹ to R⁶ is replaced by any of the other functional group(s) disclosed herein, but where the biological inertness of the polymer is not substantially altered.

In one aspect of the polyphosphazene of formula (I) illustrated above, for example, at least one of the substituents R¹ to R⁶ can be an unsubstituted alkoxy substituent, such as methoxy (OCH₃), ethoxy (OCH₂CH₃) or n-propoxy (OCH₂CH₂CH₃). In another aspect, for example, at least one of the substituents R¹ to R⁶ is an alkoxy group substituted with at least one fluorine atom. Examples of useful fluorine-substituted alkoxy groups R¹ to R⁶ include, but are not limited to OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CF₂, OCH₂(CF₂)₄CHF₂, OCH₂(CF₂)₅CHF₂, OCH₂(CF₂)₆CHF₂, OCH₂(CF₂)₇CHF₂, and the like. Thus, while trifluoroethoxy (OCH₂CF₃) groups are preferred, these further exemplary functional groups also may be used alone, in combination with trifluoroethoxy, or in combination with each other. In one aspect, examples of especially useful fluorinated alkoxide functional groups that may be used include, but are not limited to, 2,2,3,3,3-pentafluoropropyloxy (OCH₁₂CF₂CF₃), 2,2,2,2′,2′,2′-hexafluoroisopropyloxy (OCH(CF₃)₂), 2,2,3,3,4,4,4-heptafluorobutyloxy (OCH₂CF₂CF₂CF₃), 3,3,4,4,5,5,6,6,7,7,8,8,8-tridecafluorooctyloxy (OCH₂(CF₂)₇CF₃), 2,2,3,3,-tetrafluoropropyloxy (OCH₂CF₂CHF₂), 2,2,3,3,4,4-hexafluorobutyloxy (OCH₂CF₂CF₂CHF₂), 3,3,4,4,5,5,6,6,7,7,8,8-dodecafluorooctyloxy (OCH₂(CF₂)₇CHF₂), and the like, including combinations thereof.

Further, in some embodiments, 1% or less of the R¹ to R⁶ groups may be alkenoxy groups, a feature that may assist in crosslinking to provide a more elastomeric phosphazene polymer. In this aspect, alkenoxy groups include, but are not limited to, OCH₂CH═CH₂, OCH₂CH₂CH═CH₂, allylphenoxy groups, and the like, including combinations thereof: Also in formula (I) illustrated herein, the residues R¹ to R⁶ are each independently variable and therefore can be the same or different.

By indicating that n can be as large as ∞ in formula I where R1 through R⁶ are all trifluoroethoxy (OCH₂CF₃) groups, it is intended to specify values of n that encompass polyphosphazene polymers that can have an average molecular weight of up to about 75 million Daltons. In one aspect for example, n of formula I can vary from at least about 2,700 to about 100,000. In another aspect, by indicating that n can be as large as ∞ in formula I, it is intended to specify values of n from about 4,000 to about 50,000, more preferably, n is about 7,000 to about 40,000 and most preferably n is about 13,000 to about 30,000.

In the present invention, the polyphosphazene polymers disclosed herein have molecular weights of at least about 2,000,000 g/mol, at least about 3,000,000 g/mol, at least about 4,000,000 g/mol, or at least about 5,000,000 g/mol, with preferred polymers having molecular weights of at least about 10,000,000 g/mol. In the present invention, polyphosphazene polymers having molecular weights of less than about 2,000,000 often display kinetic or mechanical characteristics that make them less suitable for uses in applications according to the present invention.

In one aspect, any R¹ to R⁶, whether that R¹ to R⁶ is halogenated or substituted in any way or not, typically has up to about 20 carbon atoms. In another aspect, any R¹ to R⁶, whether that R¹ to R⁶ is halogenated or substituted in any way or not, may independently have up to about 20 carbon atoms, up to about 15 carbon atoms, up to about 12 carbon atoms, or up to about 10 carbon atoms. However, when any R¹ to R⁶ is a dialkylamino, the number of carbon atoms in this moiety can be higher because each alkyl group of the dialkylamino can have up to 20 carbon atoms.

Furthermore, the certain polyphosphazene polymers of the present invention typically may be selected so that at least one of the groups R¹ to R⁶ in the polymer is preferably an alkoxy group substituted with at least one fluorine atom.

The alkyl group in the alkoxy, alkylsulfonyl and dialkyl amino groups include straight or branched chain alkyl groups with 1 to 20 carbon atoms, wherein the alkyl groups may be substituted with at least one halogen atom, such as a fluorine atom.

Examples of alkoxy groups include methoxy, ethoxy, propoxy and butoxy groups, which preferably can be substituted with at least one fluorine atom. Particularly preferred is the 2,2,2-trifluoroethoxy group. Examples of alkylsulfonyl groups are methyl, ethyl, propyl and butylsulfonyl groups. Examples of dialkyl amino groups are the dimethyl, diethyl, dipropyl, and dibutylamino groups.

The aryl group in the aryloxy group is, for example, a compound with one or more aromatic ring systems, wherein the aryl group can be substituted with at least one of the previously defined alkyl groups, for example. Examples of aryloxy groups are phenoxy and naphthoxy groups and derivatives thereof.

The heterocyclic alkyl group is for example a 3 or 7 membered ring system wherein at least one ring atom is a nitrogen atom. The heterocyclic alkyl group can, for example, be substituted by at least one of the previously defined alkyl groups. Examples of heterocyclic alkyl groups include piperidinyl, piperazinyl, pyrrolidinyl and morpholinyl groups and derivatives thereof. The heteroaryl group can be a compound with one or more aromatic ring systems, wherein at least one ring atom is a nitrogen atom. The heteroaryl group can be substituted with at least one of the previously defined alkyl groups, for example. Examples of heteroaryl groups include pyrrolyl, pyridinyl, pyridinoyl, isoquinolinyl, and quinolinyl groups and derivatives thereof.

In a preferred embodiment of the present invention, the biocompatible coating contains the antithrombogenic polymer poly[bis(trifluoroethoxy)phosphazene].

It was found, surprisingly, that the polymer of formula (I) defined above exhibits outstanding matrix properties for pharmacologically active agents, such that the polymer serves to hold, contain, embed, and/or immobilize the pharmacologically active agents, and these agents subsequently can be delivered to their surroundings in a controlled or measured fashion as the polymer matrix releases them. In one aspect, an implant material serves as a substrate for the polymer of formula (I) which coats at least a portion of the substrate, and at least one pharmacologically active agent is contacted with the polymer coated implant such that the polymer matrix takes up and then holds or immobilizes the pharmacologically active agent(s), to be released when implanted. In another aspect, an implant material serves as a substrate which is contacted at substantially the same time with a polymer of formula (I) and at least one pharmacologically active agent, typically in solution, such that the pharmacologically active agent is contained in, immobilized in, associated with, or embedded in the polymer matrix as it coats at least a portion of the substrate, again to be released when implanted. It was also found, surprisingly, that there is no inflammatory reaction resulting from the contact of the polymer of formula (I) with tissue, whether before, during, or after the controlled release of a pharmacologically active agent from the polymer. This feature makes possible a controlled release of active agent through active agent diffusion and dissolution, without the occurrence of an undesired inflammatory reaction.

Because of the non-reactive nature of the polymer of formula (I), the pharmacologically active agent to be used in the implant does not exhibit any specific limitations, and is preferably an organic (low or higher molecular weight) compound, especially an antimitogenic active agent such as a cytostatic (such as rapamycin, paclitaxel or taxol, respectively, etc.), a PDGF-inhibitor (such as tyrphostins etc.), a Raf-1 kinase inhibitor, a monoclonal antibody for integrin blockade of leukocytes, an antisense active agent (such as plasmid DNA, antisense-RNA etc.), superoxide dismutase, a radical trap (such as probucol etc.), a steroid, a statin (such as cerivastatin etc.), a corticosteroid (such as methotrexate, dexamethasone, methylprednisolone, etc.), an adenylate cyclase inhibitor (such as forskolin etc.), a somatostatin analogue (such as angiopeptin etc.), an antithrombin agent (such as argatroban etc.), a nitric oxide donor, a glycoprotein IIb/IIIa receptor antagonist (such as urokinase derivatives, abciximab, tirofiban etc.), an antithrombotic agent (such as activated protein C, PEG-hirudin, prostaglandin analogues etc.), a vascular endothelial growth factor (VEGF), trapidil etc., and mixtures of these. In a preferred embodiment of the present invention, the pharmacologically active agent is tacrolimus, genexol, paclitaxel or taxol (cf. R. T. Liggins, W. L. Hunter and H. M. Burt, Journal of Pharmaceutical Sciences, Vol. 86, No. 12, 1997). By using said pharmacologically active agents (alone or in a mixture), a homogenous and stable mixture in the polymer having the formula (I), preferably in poly[bis(trifluoroethoxy)phosphazene], can be obtained.

In one aspect, it is desirable that the content of pharmacologically active agent(s) in the implant according to the present invention is as high as possible to e.g. prevent disorders caused by the implant such as restenosis, effectively. The weight ratio (mixing ratio) of the polymer having the formula (I) to the pharmacologically active agent(s) can be from about 10,000:1 to about 1:1. For example, the weight ratio of polymer of formula (I) to the pharmacologically active agent(s) can be from about 100:1 to about 1:1, from about 50:1 to about 1:1, 20:1 to about 1:1, or from about 10:1 to about 1:1. Preferably, this weight ratio (mixing ratio) of the polymer (I) to the active agent(s) can be from about 5:1 to about 1:1. In this context, it is preferred that the polymer (I) and the pharmacologically active agent(s) are miscible in each other and result in a homogenous and stable matrix material, and should preferably not result in a phase separation.

In a further aspect, it is desirable that the content of active agent in the biocompatible coating be as high as possible to prevent restenosis effectively. It has been shown that the coating may contain up to 50% by weight of active agent without significant damage to the mechanical properties of the coating. According to the invention, the proportion of active agent in the coating is in the range of 0.01 to 50% by weight, and preferably 0.2 to 30% by weight. This is approximately equivalent to a polymer to active agent weight ratio of 1:0.000 to 1 to 1:1, preferably 1:0.05 to 1:0.5.

The biocompatible coating of the artificial implant according to the invention has, for example, a thickness of 1 nm to about 100 μm, preferably 10 nm to 10 μm, and especially preferred up to about 1 μm.

There is no particular limit to the implant material used as a substrate according to the invention. It may be any implant material such as plastics, metals, metal alloys and ceramics. For example, the implant material can be an artificial heart valve of pyrolyzed carbon or a stent such as is described in DE-A-197 53 123.

In one embodiment of an artificial implant according to the invention there may be a layer containing an adhesion promoter provided between the surface of the substrate and the biocompatible coating.

The adhesion promoter or spacer in an embodiment of an artificial implant according to the invention may be, for example, an organosilicon compound, preferably an amino-terminated silane or a compound based on an aminosilane, or an alkylphosphonic acid. Aminopropyltrimethoxysilane is especially preferred.

An adhesion promoter in an embodiment of an artificial implant according to the invention particularly improves the adhesion of the coating to the surface of the implant material through coupling of the adhesion promoter to the surface of the implant material, through, for instance, ionic and/or covalent bonds, and through further coupling of the adhesion promoter to reactive components, particularly to the antithrombogenic polymer of the coating, through, for instance, ionic and/or covalent bonds.

In addition, a process for producing the artificial implants according to the invention is provided, wherein the biocompatible coating is applied to the substrate by reacting the substrate with
(a) a mixture of the antithrombogenic polymer or a precursor of it and the active agent or
(b) the antithrombogenic polymer or a precursor of it to produce a primary polymer coating, and subsequent application/penetration of the active agent into the primary polymer coating.

Especially preferred is a wet chemical process, particularly for process variant (a), because the active agent is often sensitive to drastic reaction conditions. In this case, the substrate is immersed in a solution containing the antithrombogenic polymer and active agent, and optionally the solvent is then removed either by heating or by applying a vacuum, This process is repeated until the coating has the desired thickness.

Suitable solvents for this process are selected from polar aprotic solvents such as esters (such as ethyl acetate, propyl acetate, butyl acetate, ethyl propionate, ethyl butyrate etc.), ketones (such as acetone, ethyl methyl ketone etc.), amides (such as dimethylformamide etc.), sulfoxides (such as DMSO etc.) and sulfones (such as sulfolane etc.). Ethyl acetate is especially preferred. The concentration of the polymer in the solution is 0.001 to 0.5 M, preferably 0.01 to 0.1 M. The concentration of the active agent depends on the desired ratio of polymer to active agent. The immersion time is preferably in the range of 10 seconds to 100 hours. The drying steps are done in vacuum, in air, or in a protective gas in the temperature range, for example, from about −20° C. to about 300° C., preferably 0° C. to 200° C., and especially preferably from 20° C. to 100° C.

The other processes mentioned in DE 196 13 048 can also be used for stable active agents, such as the process of applying polydichlorophosphazene and subsequent reaction with reactive compounds, of melting on, or of sublimation. These processes are usable particularly for the first step of process variant (b), in which the active agent is applied or penetrates in a second step, which second step can then be done preferably by a gentle wet chemical method such as is described above.

In the process using polydichlorophosphazene, a mixture of polydichlorophosphazene and active agent is applied to the surface of the substrate and reacted with at least one reactive compound selected from aliphatic or aromatic alcohols or their salts, alkylsulfones, dialkylamines, and aliphatic or aromatic heterocycles having nitrogen as the heteroatom, corresponding to the definition of R¹ to R⁶, above. The polydichlorophosphazene is preferably applied to the surface of the substrate in an inert gas atmosphere, optionally coupled to the adhesion promoter, and reacted with the reactive compound. Alternatively, polydichlorophosphazene can be applied under reduced pressure or in air, and optionally coupled to the adhesion promoter.

The production of polymers of formula (I), such as poly[bis(trifluoroethoxy)phosphazene], starting with hexachlorocyclotriphosphazene, is known at the state of the art. The polymerization of hexachlorocyclotriphosphazene is described extensively in Korsak et al., Acta Polymerica 30, No. 5, pages 245-248 (1979). Esterification of the polydichlorophosphazene produced by the polymerization is described in Fear, Thower and Veitch, J. Chem. Soc., page 1324 (1958).

In a preferred embodiment of the process according to the invention, an adhesion promoter as defined above is applied to the surface of the substrate before application of the mixture of polymer or polymer precursor and active agent, or before application of polymer or polymer precursor, and coupled to the surface through ionic and/or covalent bonds, for instance. Then the antithrombogenic polymer of polydichlorophosphazene, for example, is applied to the substrate surface coated with the adhesion promoter and is coupled to the adhesion promoter through ionic and/or covalent bonds, for instance.

The adhesion promoter can be applied to the substrate by wet chemistry or in solution or from the melt or by sublimation or spraying. The wet chemical coupling of an adhesion promoter based on amino acids on hydroxylated surfaces, is described in the diploma thesis of Marco Mantar, page 23, University of Heidelberg (1991).

The substrate surface can be cleaned oxidatively, with Caro's acid, for instance, before application of polydichlorophosphazene, the adhesion promoter, or the antithrombogenic polymer. Oxidative cleaning of surfaces with simultaneous hydroxylation, such as can be used, for instance, for implants of plastics, metals or ceramics, is described in Ulman Abraham, Analysis of Surface Properties, “An Introduction to Ultrathin Organic Films”, 108, 1991.

In summary, it has been established that the artificial implants according to the invention surprisingly retain the outstanding mechanical properties of the implant material as the substrate. Due to the coating applied according to the invention, for instance, by direct deposition from the solution, they exhibit not only antithrombogenic but also anti-restenosis properties, drastically improving the biocompatibility and usability of such artificial implants. These surprising results can be demonstrated easily by X-ray photoelectron (XPS) spectra.

The present invention is further illustrated in the following examples. The following examples are put forth so as to provide those of ordinary skill in the art with a complete disclosure and description of the claimed methods, and is intended to be purely exemplary of the invention and is not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in degree C. and pressure is at or near atmospheric.

### EXAMPLES

### Example 1

A: The polydichlorophosphazene on which the poly[bis(trifluoroethoxy)phosphazene] is based, is produced by polymerization of hexachlorocyclotriphosphazene at 250±1° C. in an ampule with a diameter of 5.0 mm and under a pressure of 1.3 Pa (10² mm Hg) prevailing in the ampule. This is done by first preparing a 0.1 M solution of polydichlorophosphazene (0.174 g in 5 ml solvent) in an inert gas atmosphere. Absolute toluene is used as the solvent. Then the esterification is done in this solution with sodium 2,2,2-trifluoroethanolate in absolute tetrahydrofuran as the solvent (8 ml absolute tetrahydrofuran, 0.23 g sodium, 1.46 ml 2,2,2-trifluoroethanol).

B; For oxidative cleaning and simultaneous hydroxylation of the artificial implant surfaces, the substrate is placed in a mixture of 1:3 30% H₂O₂ and concentrated sulfuric acid (Caro's acid) for 2 hours at a reaction temperature of 80° C. After that treatment, the substrate is washed with 0.5 liters deionized water [with a resistivity] of 18 MΩ/cm and about pH 5, and then dried in a stream of nitrogen.

C: To coat the surface of the implant with an adhesion promoter, the artificial implant, oxidatively cleaned with Caro's acid according to Example 1B, is immersed for 30 minutes at room temperature in a 2% solution of aminopropyltrimethoxysilane in absolute ethanol. Then the substrate is washed with 4-5 ml absolute ethanol and left in the drying cabinet for 1 hour at 105° C.

### Example 2

A: An artificial implant pretreated according to Example 1B and 1C was placed for 24 hours at room temperature in a 0.1 M solution of poly[bis(trifluoroethoxy)phosphazene] in ethyl acetate (0.121 g in 5 ml ethyl acetate) which contained 0.0121 g probucol. Then the artificial implant produced in that manner was washed with 4-5 ml ethyl acetate and dried in a stream of nitrogen.

B: An artificial implant pretreated according to Example 1B and 1C was placed for 24 hours at room temperature in a 0.1 M solution of poly[bis(trifluoroethoxy)phosphazene] in ethyl acetate (0.121 g in 5 ml ethyl acetate) which contained 0.0242 g trapidil. Then the artificial implant produced in that manner was washed with 4-5 ml ethyl acetate and dried in a stream of nitrogen.

The surfaces of the artificial implants produced in Examples 2A and 2B were examined by photoelectron spectrometry to determine their elemental composition, their stoichiometry and the coating thickness. The results showed that the poly[bis(trifluoroethoxy)phosphazene] had been successfully immobilized with aminopropyltrimethoxysilane as the adhesion promoter, and that coating thicknesses greater than 2.4 nm were attained. Further, it could also be shown by analysis (NMR) that trapidil or probucol had been embedded in the coating in corresponding proportion.

### Example 3

An artificial implant cleaned according to Example 1B was placed for 24 hours at 70° C. in a 0.1 M solution of poly[bis(trifluoroethoxy)phosphazene] in ethyl acetate (0.121 g in 5 ml ethyl acetate) which contained 0.0121 g probucol. Then the artificial implant treated in that manner was washed with 4-5 ml ethyl acetate and dried in a stream of nitrogen.

The artificial implant prepared in this manner was examined by photoelectron spectrometry to determine its elemental composition, its stoichiometry, and the coating thickness. The results showed that the poly[bis(trifluoroethoxy)phosphazene] had been coupled to the implant surface and coating thicknesses greater than 2.1 mm were attained. Further, it could also be shown that the probucol was embedded in the coating in corresponding proportion.

### Example 4

A: An artificial implant pretreated according to Example 1B and 1C was placed for 24 hours at room temperature in a 0.1 M solution of poly[bis(trifluoroethoxy)phosphazene] in ethyl acetate (0.121 g in 5 ml ethyl acetate). Then the artificial implant prepared in this manner was washed with 4-5 ml ethyl acetate and dried in a stream of nitrogen

B; The substrate obtained according to Example 4A was immersed for 24 hours at room temperature in a solution of cerivastatin in ethyl acetate (0.0121 g cerivastatin in 5 ml ethyl acetate). After drying in a stream of nitrogen, it was shown analytically that the layer of poly[bis(trifluoroethoxy)phosphazene] contained cerivastatin.

It will be appreciated by those possessing ordinary skill in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An implant comprising:
a) a substrate comprising an implant material,
b) a biocompatible matrix coating at least part of the surface of the substrate, and
c) at least one pharmacologically active agent associated with the biocompatible matrix;
wherein:
the biocompatible matrix comprises an antithrombogenic polymer having the formula 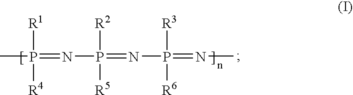
R¹ to R⁶ are the same or different and are selected independently from an alkoxy, an alkylsulfonyl, a dialkylamino, an aryloxy, a heterocycloalkyl group having nitrogen as the heteroatom, a heteroaryl group having nitrogen as the heteroatom, or a halogenated derivative thereof, any of which independently having up to 20 carbon atoms; and
n may vary from about 40 to about 100,000.

2. An implant according to claim 1, wherein at least one of the groups R¹ to R⁶ is an alkoxy group substituted with at least one fluorine atom.

3. An implant according to claim 1, wherein the antithrombogenic polymer is poly[bis(trifluoroethoxy)phosphazene].

4. An implant according to claim 1, wherein the pharmacologically active agent is selected from an antimitogenic agent, a cytostatic agent, a PDGF-inhibitor, a Raf-1 kinase inhibitor, a monoclonal antibody for integrin blockade of leukocytes, an antisense active agent, a superoxide dismutase, a radical trap, a steroid, a statin, a corticosteroid, an adenylate cyclase inhibitor, a somatostatin analogue, an antithrombin agent, a nitric oxide donor, a glycoprotein IIb/IIIa receptor antagonist, an antithrombotic agent, a prostaglandin analogue, a vascular endothelial growth factor (VEGF), or any combination thereof.

5. An implant according to claim 1, wherein the pharmacologically active agent is selected from rapamycin, paclitaxel, a tyrphostin, plasmid DNA, antisense-RNA, superoxide dismutase, probucol, cerivastatin, methotrexate, dexamethasone, methylprednisolone, forskolin, angiopeptin, argatroban, a urokinase derivative, abciximab, tirofiban, activated protein C, PEG-hirudin, trapidil, tacrolimus, genexol, or any combination thereof.

6. An implant according to claim 1, wherein the weight ratio of antithrombogenic polymer to pharmacologically active agent is from 1:0.0001 to 1:1.

7. An implant according to claim 1, further comprising an adhesion promoter situated between the substrate and the biocompatible matrix.

8. An implant according to claim 7, wherein the adhesion promoter is an amino-terminated silane.

9. A method for making an implant, comprising:
a) providing a substrate comprising an implant material, a biocompatible matrix, and at least one pharmacologically active agent; and
b) either:
i) contacting the substrate with a mixture of the biocompatible matrix and the at least one pharmacologically active agent; or
ii) contacting the substrate with the biocompatible matrix to produce a primary polymer-coated substrate, following by contacting the primary polymer-coated substrate with the at least one pharmacologically active agent;
wherein:
the biocompatible matrix comprises an antithrombogenic polymer having the following formula (I) or a precursor to formula (I) 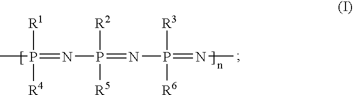
R¹ to R⁶ are the same or different and are selected independently from an alkoxy, an alkylsulfonyl, a dialkylamino, an aryloxy, a heterocycloalkyl group having nitrogen as the heteroatom, a heteroaryl group having nitrogen as the heteroatom, or a halogenated derivative thereof, any of which independently having up to 20 carbon atoms; and
n may vary from about 40 to about 100,000.

10. A method according to claim 9, wherein the contacting step b) occurs in a solution comprising the biocompatible matrix, the at least one pharmacologically active agent, and at least one dipolar aprotic solvent.

11. A method according to claim 10, wherein the at least one dipolar aprotic solvent comprises ethyl acetate.

12. A method according to claim 9, further comprising contacting the substrate comprising an implant material of step a) with an adhesion promoter, prior to the contacting step b).

13. A method according to claim 12, wherein the adhesion promoter is an amino-terminated silane.

14. A method according to claim 9, wherein at least one of the groups R¹ to R⁶ is an alkoxy group substituted with at least one fluorine atom.

15. A method according to claim 9, wherein the antithrombogenic polymer is poly[bis(trifluoroethoxy)phosphazene].

16. A method according to claim 9, wherein the pharmacologically active agent is selected from an antimitogenic agent, a cytostatic agent, a PDGF-inhibitor, a Raf-1 kinase inhibitor, a monoclonal antibody for integrin blockade of leukocytes, an antisense active agent, a superoxide dismutase, a radical trap, a steroid, a statin, a corticosteroid, an adenylate cyclase inhibitor, a somatostatin analogue, an antithrombin agent, a nitric oxide donor, a glycoprotein IIb/IIIa receptor antagonist, an antithrombotic agent, a prostaglandin analogue, a vascular endothelial growth factor (VEGF), or any combination thereof.

17. A method according to claim 9, wherein the pharmacologically active agent is selected from rapamycin, paclitaxel, a tyrphostin, plasmid DNA, antisense-RNA, superoxide dismutase, probucol, cerivastatin, methotrexate, dexamethasone, methylprednisolone, forskolin, angiopeptin, argatroban, a urokinase derivative, abciximab, tirofiban, activated protein C, PEG-hirudin, trapidil, tacrolimus, genexol, or any combination thereof.

18. A method according to claim 9, wherein the weight ratio of antithrombogenic polymer to pharmacologically active agent is from 1:0.0001 to 1:1.
